(19) United States
(12) Patent Application Publication
(10) Pub. No.: US 2022/0076813 A1
(43) Pub. Date: Mar. 10, 2022

(54) IMMUNIZATION REGISTRY INTEGRATION SYSTEM

(71) Applicant: MarkeTouch Media, Inc., Houston, TX (US)

(72) Inventors: Lyle M. Green, Pacific Palisades, CA (US); Matthew K. Feltman, Isle Of Palms, SC (US); Charles E. Russo, Houston, TX (US)

(21) Appl. No.: 17/228,113

(22) Filed: Apr. 12, 2021

Related U.S. Application Data
(60) Provisional application No. 63/076,326, filed on Sep. 9, 2020.

(51) Int. Cl. G16H 40/20; H04L 29/08; G06Q 10/10; G06F 21/62

(52) U.S. Cl. CPC G16H 40/20; H04L 67/18; G06F 21/6245; G06Q 10/1095; H04L 67/26

(57) ABSTRACT

Techniques are described for vaccine notification and scheduling in automated electronic delivery format. A system for immunization registry integration that supports vaccine notification and scheduling in automated electronic delivery format is provided. One method includes providing access to a scheduling application to schedule a patient appointment. The method may include receiving authorization to query an immunization registry based at least on the provided access. The method may also include determining one or more immunization opportunities based at least in part on the received authorization. Additionally, the method may communicating the one or more immunization opportunities based at least in part on the determining.

### CROSS REFERENCE

The present application for patent claims the benefit of U.S. Provisional Patent Application No. 63/076,326 by GREEN et al., entitled “IMMUNIZATION REGISTRY INTEGRATION SYSTEM,” filed Sep. 9, 2021, which is expressly incorporated by reference herein for all purposes.

### FIELD OF TECHNOLOGY

The present disclosure relates generally to database systems, and more specifically to immunization registry integration systems for patient initiated recommended vaccine notification and scheduling in automated electronic delivery format.

### DESCRIPTION OF RELATED ART

Public health organizations have been using immunization registries for over twenty years. In some cases, these public health organizations are state based. Some states require the submission of immunizations, while other states do not. Pharmacies and health care providers may have the ability to query a registry to see what immunizations a patient has received, and thus offer the patient the opportunity to be vaccinated where there are gaps. To support a pharmacy, health care provider, and/or other company's interactions with its customers and patients, systems have been developed as a centralized, scalable mechanism related to various customer contact contexts, including, for example, sales and marketing contacts, service order contacts, technical support issues, and billing questions. A cloud platform (i.e., a computing platform for cloud computing) or hosted platform (i.e., a privately-managed computing platform utilizing cloud computing) may be employed by various entities to store, manage, and process data using a network of remote servers to support these systems.

### SUMMARY

The described features generally relate to one or more improved methods, systems, or devices that provide techniques notification and scheduling in automated electronic delivery format associated with immunization registry integration systems. In some examples, a system may provide a patient and/or health care provider access to online health and wellness scheduling applications as well as outbound notifications. The system may perform immunization registry integration processes and provide notification and scheduling communications utilizing SMS/MMS text messages, voice calls (e.g., recorded live and voice mail messages), mobile device app push notification, email, etc.

The described techniques may include providing access to a scheduling application to schedule a patient appointment, receiving authorization to query an immunization registry based at least on the provided access, determining one or more immunization opportunities based at least in part on the received authorization; and communicating the one or more immunization opportunities based at least in part on the determining.

The described techniques may include providing access to a scheduling application to schedule a patient appointment, transmitting an indication of a scheduled patient appointment to a device, receiving information associated with immunization registry responsive to the transmitted indication, determining one or more immunization opportunities based at least in part on the received information associated with immunization registry, and communicate the one or more immunization opportunities based at least in part on the determining.

The described techniques may include providing access to an immunization services application, receiving authorization to query an immunization registry based at least on the provided access, receiving information associated with the immunization registry based at least in part on the received authorization, determining one or more immunization opportunities based at least in part on the received information associated with immunization registry, and communicating the one or more immunization opportunities based at least in part on the determining.

A method for immunization registry integration is described. The method may include providing access to a scheduling application to schedule a patient appointment, receiving authorization to query an immunization registry based at least on the provided access, determining one or more immunization opportunities based at least in part on the received authorization, and communicating the one or more immunization opportunities based at least in part on the determining.

An apparatus for immunization registry integration is described. The apparatus may include a processor, memory coupled with the processor, and instructions stored in the memory. The instructions may be executable by the processor to cause the apparatus to provide access to a scheduling application to schedule a patient appointment, receive authorization to query an immunization registry based at least on the provided access, determine one or more immunization opportunities based at least in part on the received authorization, and communicate the one or more immunization opportunities based at least in part on the determining.

Another apparatus for immunization registry integration is described. The apparatus may include means for providing access to a scheduling application to schedule a patient appointment, means for receiving authorization to query an immunization registry based at least on the provided access, means for determining one or more immunization opportunities based at least in part on the received authorization, and means for communicating the one or more immunization opportunities based at least in part on the determining.

A non-transitory computer-readable medium storing code for immunization registry integration is described. The code may include instructions executable by a processor to provide access to a scheduling application to schedule a patient appointment, receive authorization to query an immunization registry based at least on the provided access, determine one or more immunization opportunities based at least in part on the received authorization, and communicate the one or more immunization opportunities based at least in part on the determining.

Some examples of the method, apparatus, and non-transitory computer-readable medium described herein may further include operations, features, means, or instructions for receiving, over a network, a request to schedule the patient appointment and wherein the providing access to the scheduling application may be responsive to the request.

Some examples of the method, apparatus, and non-transitory computer-readable medium described herein may further include operations, features, means, or instructions for obtaining one or more data elements based at least in part on the request to schedule the patient appointment, querying the immunization registry based at least in part on the one or more data elements, and wherein the determining one or more immunization opportunities may be based at least in part on the querying.

In some examples of the method, apparatus, and non-transitory computer-readable medium described herein, the one or more data elements comprise an identifier corresponding to at least one of name, date of birth, or gender.

In some examples of the method, apparatus, and non-transitory computer-readable medium described herein, the one or more data elements comprise a geographical location identifier.

In some examples of the method, apparatus, and non-transitory computer-readable medium described herein, the one or more data elements comprise a plurality of phone numbers.

In some examples of the method, apparatus, and non-transitory computer-readable medium described herein, the plurality of phone numbers comprises a first phone number entered for scheduling the patient appointment and a second phone number determined from a source different from the scheduling application.

In some examples of the method, apparatus, and non-transitory computer-readable medium described herein, the one or more immunization opportunities comprises an immunization opportunity unrelated to the patient appointment.

In some examples of the method, apparatus, and non-transitory computer-readable medium described herein, the one or more immunization opportunities comprises an immunization opportunity corresponding to the patient appointment.

Some examples of the method, apparatus, and non-transitory computer-readable medium described herein may further include operations, features, means, or instructions for providing a user prompt to select the one or more immunization opportunities based at least in part on the communicating the one or more immunization opportunities.

Some examples of the method, apparatus, and non-transitory computer-readable medium described herein may further include operations, features, means, or instructions for providing access to a new appointment instance of the scheduling application to schedule the one or more immunization opportunities based at least in part on the communicating the one or more immunization opportunities.

In some examples of the method, apparatus, and non-transitory computer-readable medium described herein, the communicating the one or more immunization opportunities comprises a real-time user prompt to add the one or more immunization opportunities to the patient appointment being scheduled.

In some examples of the method, apparatus, and non-transitory computer-readable medium described herein, the communicating the one or more immunization opportunities comprises an electronic communication subsequent to the patient appointment being scheduled.

A method for immunization registry integration is described. The method may include providing access to healthcare services information, identifying information from a medical-related digital document based at least in part on provided access, receiving authorization to query an immunization registry based at least on the medical-related digital document, determining one or more immunization opportunities based at least in part on the received authorization, and communicating the one or more immunization opportunities based at least in part on the determining.

An apparatus for immunization registry integration is described. The apparatus may include a processor, memory coupled with the processor, and instructions stored in the memory. The instructions may be executable by the processor to cause the apparatus to provide access to healthcare services information, identify information from a medical-related digital document based at least in part on provided access, receive authorization to query an immunization registry based at least on the medical-related digital document, determine one or more immunization opportunities based at least in part on the received authorization, and communicate the one or more immunization opportunities based at least in part on the determining.

Another apparatus for immunization registry integration is described. The apparatus may include means for providing access to healthcare services information, means for identifying information from a medical-related digital document based at least in part on provided access, means for receiving authorization to query an immunization registry based at least on the medical-related digital document, means for determining one or more immunization opportunities based at least in part on the received authorization, and means for communicating the one or more immunization opportunities based at least in part on the determining.

A non-transitory computer-readable medium storing code for immunization registry integration is described. The code may include instructions executable by a processor to provide access to healthcare services information, identify information from a medical-related digital document based at least in part on provided access, receive authorization to query an immunization registry based at least on the medical-related digital document, determine one or more immunization opportunities based at least in part on the received authorization, and communicate the one or more immunization opportunities based at least in part on the determining.

Some examples of the method, apparatus, and non-transitory computer-readable medium described herein may further include operations, features, means, or instructions for receiving, over a network, a request to provide the medical-related digital document.

Some examples of the method, apparatus, and non-transitory computer-readable medium described herein may further include operations, features, means, or instructions for receiving, over a network, the medical-related digital document.

Some examples of the method, apparatus, and non-transitory computer-readable medium described herein may further include operations, features, means, or instructions for obtaining one or more data elements based at least in part on the information from the medical-related digital document, querying the immunization registry based at least in part on the one or more data elements, and wherein the determining one or more immunization opportunities may be based at least in part on the querying.

Some examples of the method, apparatus, and non-transitory computer-readable medium described herein may further include operations, features, means, or instructions for providing a user prompt to select the one or more immunization opportunities based at least in part on the communicating the one or more immunization opportunities.

Some examples of the method, apparatus, and non-transitory computer-readable medium described herein may further include operations, features, means, or instructions for providing access to a scheduling application to schedule the one or more immunization opportunities based at least in part on the communicating the one or more immunization opportunities.

In some examples of the method, apparatus, and non-transitory computer-readable medium described herein, the medical-related digital document comprises a response to at least one health-related question.

### BRIEF DESCRIPTION OF THE DRAWINGS

FIG. 1 illustrates an example of a system for immunization registry integration that supports vaccine notification and scheduling in automated electronic delivery format in accordance with aspects of the present disclosure.

FIG. 2 illustrates an example of a system for immunization registry integration that supports vaccine notification and scheduling in automated electronic delivery format in accordance with aspects of the present disclosure.

FIG. 3 shows a block diagram of an apparatus associated with a system for immunization registry integration that supports vaccine notification and scheduling in automated electronic delivery format system in accordance with aspects of the present disclosure.

FIG. 4 shows a block diagram of an immunization registry integration manager that supports vaccine notification and scheduling in automated electronic delivery format in accordance with aspects of the present disclosure.

FIG. 5 shows a diagram of a system including a device associated with a system for immunization registry integration that supports vaccine notification and scheduling in automated electronic delivery format in accordance with aspects of the present disclosure.

FIGS. 6-8 show flowcharts illustrating methods associated with a system for immunization registry integration that supports vaccine notification and scheduling in automated electronic delivery format in accordance with aspects of the present disclosure.

### DETAILED DESCRIPTION

In accordance with some aspects of the present disclosure, a system may perform immunization registry integration processes, for example, so that opportunities to be vaccinated where there are gaps in immunization records may be provided to and available at the consumer level. In some examples a scheduler system (as well as other system communication modes) may be combined or integrated with real-time access to the available immunization registries. A system for immunization registry integration has the ability to provide valuable health information (for immunizations and health tests) to patients, pharmacies, health care providers, etc. The system for immunization registry integration can provide seamless access to information, appointment scheduling convenience, and improved population health.

A system for immunization registry integration may collect consumer/patient demographic data needed to access and query all available local, state and/or federal immunization registries. In some cases, the system for immunization registry integration may access at least some information via a selected immunization registry/services partner system. Based upon the information and/or data returned by the selected immunization registry/services partner system or server, the system for immunization registry integration may provide the consumer/patient a listing of any recommended and needed immunizations. This determination may be made based at least in part on the consumer/patient's past immunization history available via registry and/or the U.S. Centers for Disease Control guidance based at least in part on consumer/patient supplied demographics. Additionally or alternatively, the system for immunization registry integration may provide the ability for the consumer/patient to schedule appointments for such needed immunizations (e.g., at a designated facility provided by an immunization registry/services partner, health care provider, or the like) while electronically and digitally providing supporting documentation for any customer/patient selected immunizations. In some examples, one or more of these processes associated with the system for immunization registry integration that supports vaccine notification and scheduling in automated electronic delivery format process may be extended to available health test (e.g., COVID-19) registries.

Inventive aspects of the subject technology provide one or more solutions to various problems associated with immunization information and vaccination opportunities. Benefits of the inventive system for immunization registry integration include, but are not limited to: (i) increasing patient awareness to appropriate and personalized immunization guidelines; (ii) providing consumers the convenience of online immunization history and forecasting; (iii) improving adherence to immunization recommendations; and (iv) lowering the overall cost of healthcare through improved patient literacy, access and increased community-based vaccinations, thus reducing rates of sickness and death related to preventable disease.

In some examples, one or more servers may provide access to a scheduling application to schedule a patient appointment. The one or more servers may receive authorization to query an immunization registry based at least on the provided access. The one or more servers may determine one or more immunization opportunities based at least in part on the received authorization. The one or more servers may communicate the one or more immunization opportunities based at least in part on the determining. In some example, one or more servers may provide access to a scheduling application to schedule a patient appointment. The one or more servers may transmit an indication of a scheduled patient appointment to a device. In some cases, the device may by a patient device. The one or more servers may receive information associated with immunization registry responsive to the transmitted indication. The one or more servers may determine one or more immunization opportunities based at least in part on the received information associated with immunization registry. The one or more servers may communicate the one or more immunization opportunities based at least in part on the determining. In some examples, one or more servers may provide access to an immunization services application. The one or more examples may receive authorization to query an immunization registry based at least on the provided access. The one or more servers may receive information associated with the immunization registry based at least in part on the received authorization. The one or more servers may determine one or more immunization opportunities based at least in part on the received information associated with immunization registry. The one or more servers may communicate the one or more immunization opportunities based at least in part on the determining.

In some implementations, the techniques provided herein may be performed by one or more servers by a single entity. In some implementations, the techniques provided herein may be performed by one or more servers of multiple entities (e.g., an entity providing a consumer preference and maintenance interface and one or more entities providing health care or immunization registry services).

The one or more servers may communicate vaccine notifications and scheduling in automated electronic delivery format (e.g., a patient appointment, one or more immunization opportunities, etc.) using various techniques. In some examples, the one or more servers may identify a text-capable contact number associated with the information associated with the patient and transmit a message (e.g., SMS/MMS text message) via the text-capable contact number to communicate the one or more immunization opportunities. In some examples, the one or more servers may identify that a device associated with the patient (e.g., a patient or user account) comprises an application. For example, the device associated with the patient may include an iOS or Android-based application thereby enabling the device to operate as a client of the one or more servers, or otherwise communicate with the one or more servers. The one or more servers may transmit a push notification or message via the device to communicate the one or more immunization opportunities based at least in part on the determining that such immunization opportunities exist. In some examples, the one or more servers may identify a contact number associated with the information for providing contact associated with the patient. The one or more servers may place an outbound call (to the contact number and provide an automated message (e.g., an Interactive Voice Response (IVR) audio message) to communicate the one or more immunization opportunities. In some examples, the one or more servers (or an IVR component associated therewith) may receive an incoming call from an inbound number. The one or more servers may identify the patient based at least in part on the inbound number and the information associated with the patient. The one or more servers may provide an automated message (e.g., an IVR audio message) at some time during the inbound call to communicate the one or more immunization opportunities, a request for authorization to query an immunization registry, etc.

Aspects of the disclosure are initially described in the context of an environment supporting a database. A server may access the database to provide immunization registry integration. Aspects of the disclosure are further illustrated by and described with reference to apparatus diagrams, system diagrams, and flowcharts that relate to immunization registry integration.

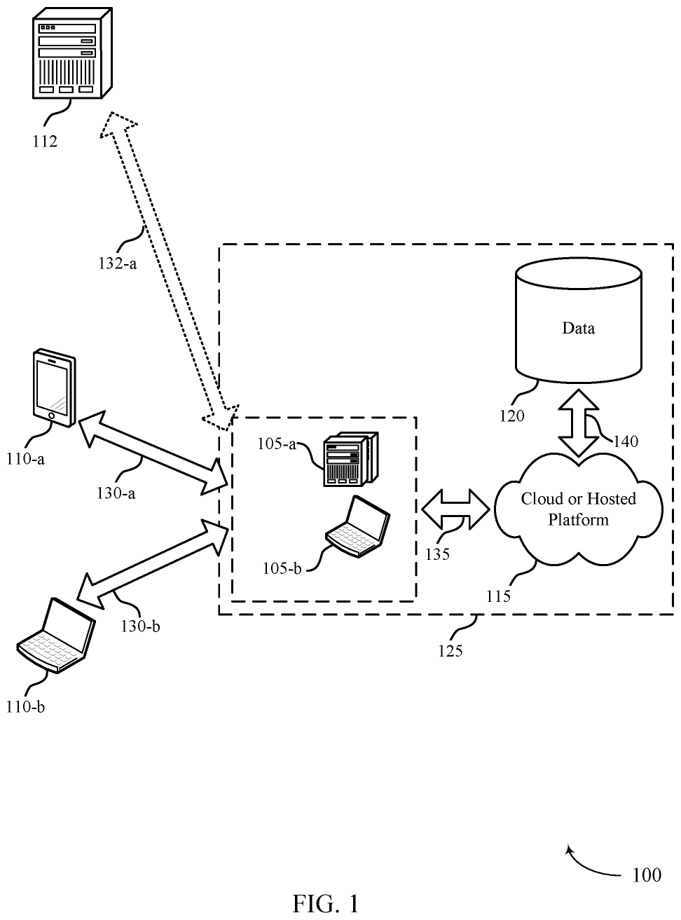

FIG. 1 illustrates an example of a system 100 for cloud computing associated with a system for immunization registry integration that supports vaccine notification and scheduling in automated electronic delivery format in accordance with aspects of the present disclosure. The system 100 includes client 105 (e.g., a cloud client or hosted client), user device contacts 110, remote system contacts 112, cloud platform 115, and data access 120. Cloud platform 115 may be an example of a public or private cloud network. A client 105 may access cloud platform 115 over network connection 135. The network may implement transfer control protocol and internet protocol (TCP/IP), such as the Internet, or may implement other network protocols. A client 105 may be an example of a user device, such as a server (e.g., client 105-a) or a laptop (e.g., client 105-b). In other examples, a client 105 may be a desktop computer, a tablet, or another computing device or system capable of generating, analyzing, transmitting, or receiving communications. In some examples, a client 105 may be operated by a user entity that is part of a business or other organization type (e.g., an entity responsible for providing consumer contact).

A client 105 may interact with multiple user device contacts 110. The interactions 130 may include communications, opportunities, purchases, sales, or any other interaction between a client 105 and a user device contact 110. Data may be associated with the interactions 130. A client 105 may access cloud or hosted platform 115 to store, manage, and process the data associated with the interactions 130. In some cases, the client 105 may have an associated security or permission level. A client 105 may have access to certain applications, data, and database information within cloud or hosted platform 115 based on the associated security or permission level, and may not have access to others.

Client 105 may interact with user device contacts 110 via text messaging, email, voice call, or any other appropriate form of interaction (e.g., interactions 130-a, or 130-b). In some examples, the interaction 130 may be a business-to-consumer (B2C) interaction. A user device contact 110 may also be referred to as or associated with a consumer, a customer, or some other suitable terminology. In some cases, the user device contact 110 may be an example of a user device, such as a mobile device (e.g., user device contact 110-a) or a laptop (e.g., user device contact 110-b). In other cases, the user device contact 110 may be another computing device that a consumer may own capable of electronic communication. In some cases, the user device contact 110 may be operable by a consumer or user authorized to access a user account.

Client 105 may also interact with remote system contacts 112 via application programming interface (API), web communication, or any other appropriate form of interaction or computing interface (e.g., interactions 132-a). In some examples, the interaction 130 may be a business-to-business (B2B) interaction. A remote system contact 112 may also be referred to as a third-party system, third-party entity, or some other suitable terminology. In some cases, the remote system contacts 112 may be an example of a server at a location different from a location of client 105. In some cases, the remote system contact 112 may be operated by one or more users or an entity different from users or an entity associated with client 105. In other cases, the remote system contact 112 may be operated by the same users or entity as the those associated with client 105.

Cloud or hosted platform 115 may provide data access or database service for the client 105. In some cases, cloud or hosted platform 115 may be an example of a single-tenant or multi-tenant database system. However, other types of systems may be implemented, including—but not limited to—client-server systems, mobile device systems, and mobile network systems. In some cases, cloud or hosted platform 115 may support customer relationship management (CRM) solutions. In some examples, the CRM solutions may include support for consumer contact, order and service fulfillment, marketing, etc. Cloud or hosted platform 115 may receive data associated with contact interactions 130 from the client 105 over network connection 135. Cloud or hosted platform 115 may receive data associated with contact interactions 132 from the client 105 over network connection 135. In some cases, cloud or hosted platform 115 may receive data directly from an interaction 130 between a user device contact 110 and the client 105. In some cases, the user device contact 110 may run an application that includes communication with client 105 and/or cloud or hosted platform 115. Cloud or hosted platform 115 may be implemented using remote servers. In some cases, the remote servers may be located at one or more data centers 120.

Data center 120 may include multiple servers. The multiple servers may be used for data storage, management, and processing. Data center 120 may receive data from cloud platform 115 via connection 140, or directly from the cloud client 105 or an interaction 130 between a contact 110 and the cloud client 105. Data center 120 may utilize multiple redundancies for security purposes. In some cases, the data stored at data center 120 may be backed up by copies of the data at a different data center (not pictured).

Subsystem 125 may include cloud clients 105, cloud platform 115, and data center 120. In some cases, data processing may occur at any of the components of subsystem 125, or at a combination of these components. In some cases, servers may perform the data processing. The servers may be a cloud client 105 or located at data center 120.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

In accordance with some implementations, client 105 (e.g., one or more consumer preference and maintenance interface servers) may comprise a system for immunization registry integration that supports vaccine notification and scheduling in automated electronic delivery format. Client 105 may provide access to a scheduling application to schedule a patient appointment. Client 105 may receive authorization to query an immunization registry based at least on the provided access. In some cases, the query of the immunization registry may be associated with a remote system contact 112 (e.g., an immunization registry/services partner software system). The client 105 may determine one or more immunization opportunities based at least in part on the received authorization. In some cases, the one or more immunization opportunities may be determined in conjunction with the remote system contact 112. The client 105 may communicate the one or more immunization opportunities based at least in part on the determining.

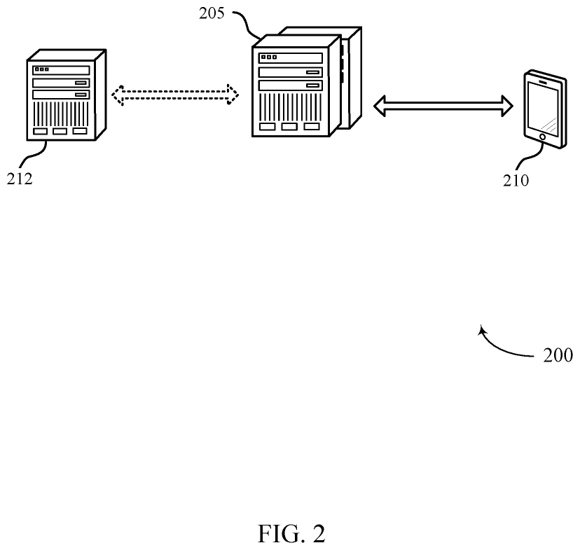

FIG. 2 illustrates an example of a system for immunization registry integration 200 that supports vaccine notification and scheduling in automated electronic delivery format in accordance with aspects of the present disclosure. In the example of FIG. 2, a client 205 (e.g., one or more consumer preference and maintenance interface servers, one or more pharmacy management software system servers, a combination of one or more consumer preference and maintenance interface servers and one or more pharmacy management software system servers, etc.) associated with a system for immunization registry integration that supports vaccine notification and scheduling in automated electronic delivery format in accordance with aspects of the present disclosure. In some cases, client 205 may communicate with and/or operate in conjunction with immunization registry/services partner software system 212 in performing one or more techniques described herein. In some cases, client 205 may communicate with user device contact 210 (e.g., a user device associated with a patient/consumer) in performing one or more techniques described herein.

A first non-limiting example of a system for immunization registry integration comprises a web-based health and wellness scheduling application-based query. Client 205 may provide access to a scheduling application to schedule a patient appointment. For example, a patient/consumer may access a web-based scheduling application to book an appointment with the patient's health care provider. During the online appointment enrollment process, client 205 may collect patient/consumer demographic data elements (i.e., in accordance with relevant privacy regulations). In some cases, the patient/consumer demographic data elements may include, but are not limited to, patient name (e.g., first, middle, last), date of birth (e.g., mm/dd/yyyy), gender, mother's maiden name, address list (e.g., street, city, state, zip code), phone number list (e.g., npa-nxx-xxxx), etc. In some cases, a minimum required patient/consumer demographic data elements may constitute one or more of these patient/consumer demographic data elements. In some cases, additional patient/consumer demographic data elements may be required and/or may be collected and utilized during the techniques and process options described herein.

Client 205 may receive authorization to query an immunization registry based at least on the provided access. For example, using the data provided by the patient/consumer and collected by client 205 during the scheduling application, client 205 may reformat the data into an electronic file and transmits that data via an application programming interface (API) or secure file transfer protocol (sFTP). In some cases, client 205 transmits that data to immunization registry/services partner software system 212. This data file sent by client 205 may include a ‘History with Forecast Request’ to the immunization registry/services partner software system 212. In some cases, immunization registry/services partner software system 212 captures and interprets the data provided via the API or sFTP. The immunization registry/services partner software system 212 may access the appropriate local, state or Federal registry (e.g., based at least in part on the demographic data of the provider or patient/consumer). Client 205 and/or immunization registry/services partner software system 212 may match the submitted patient/consumer demographic data with that currently populated/present at the local, state, Federal registry.

Client 205 may determine one or more immunization opportunities based at least in part on the received authorization. For example, if there is a one to one match of patient/consumer demographic data, the immunization registry/services partner software system 212, using associated processes and protocols may return to client 205 via the same sFTP or API a ‘History with Forecast Response,’ which may include one or more reported vaccines administered to the patient/consumer and any CDC recommended (i.e., gap) vaccines that the patient/consumer should consider. If there is not a patient/consumer match or greater than one patient/consumer demographic data set matches the demographic set information submitted by client 205, the immunization registry/services partner software system 212 may return via the same sFTP or API a listing of general vaccines recommended for the patient/consumer according to the CDC based on certain demographic information (e.g., for the gender and age submitted). In some cases, client 205 may reformat the electronic data file information provided by the immunization registry/services partner software system 212. In some cases, client 205 may personalize the returned immunization information to the patient/consumer via the web-based scheduler application or other communication methods.

Client 205 may communicate the one or more immunization opportunities based at least in part on the determining. For example, if there are one or more immunization opportunities available to the patient/consumer, client 205 may provide the patient/consumer (e.g., via the web-based scheduler application or via user device contact 210) the option to either add the registry-recommended one or more immunizations to any existing scheduled provider appointment or to schedule a new appointment for the registry recommended one or more immunizations.

A second non-limiting example of a system for immunization registry integration comprises an email appointment confirmation-based query. Client 205 may provide access to a scheduling application to schedule a patient appointment. For example, a patient/consumer may access a web-based scheduling application of client 205 to book an appointment with the patient/consumer's health care provider. Client 205 may transmit an indication of a scheduled patient appointment to user device contact 210. That is, upon completion of the appointment scheduling by the patient/consumer, client 205 transmits the patient/consumer a communication (e.g., an email) confirming information related to the patient appointment (e.g., the date, time, location and type/purpose of the scheduled appointment). In a portion of the communication (e.g., the body of the confirmation email) transmitted to the patient/consumer, client 205 may imbed a Uniform Resource Locator (URL). According to some aspects, a purpose of the URL is to capture patient/consumer authorization of a query of an immunization registry tool. That is, the patient/consumer authorization may confirm the patient/consumer's interest in learning about other vaccines for which he or she may be eligible.

Client 205 may receive information associated with immunization registry responsive to the transmitted indication. When selected by the patient/consumer, the URL may redirect the patient/consumer to a website that will collect patient/consumer demographic data elements. For example, client 205 may collect patient/consumer demographic data elements (i.e., in accordance with relevant privacy regulations) as discussed herein. Using the data provided by the consumer and collected by client 205 (and/or immunization registry/services partner software system 212), client 205 may reformat the data into an electronic file and transmits that data via an API or sFTP. In some cases, a ‘History with Forecast Request’ may be provided to the immunization registry/services partner software system 212. In some examples, the immunization registry integration associated with one or both of the client 205 or immunization registry/services partner software system 212 enables the capture and interpretation of the data provided via the API or sFTP.

Client 205 may determine one or more immunization opportunities based at least in part on the received information associated with immunization registry. In some examples, one or both of the client 205 or immunization registry/services partner software system 212 may access the appropriate local, state or Federal registry (e.g., based at least in part on the demographic data of the provider or patient/consumer). For example, if there is a one to one match of patient/consumer demographic data, the immunization registry/services partner software system 212, using associated processes and protocols may return to client 205 via the same sFTP or API a ‘History with Forecast Response,’ which may include one or more reported vaccines administered to the patient/consumer and any CDC recommended (i.e., gap) vaccines that the patient/consumer should consider. If there is not a patient/consumer match or greater than one patient/consumer demographic data set matches the demographic set information submitted by client 205, the immunization registry/services partner software system 212 may return via the same sFTP or API a listing of general vaccines recommended for the patient/consumer according to the CDC based on certain demographic information (e.g., for the gender and age submitted). In some cases, client 205 may reformat the electronic data file information provided by the immunization registry/services partner software system 212. In some cases, client 205 may personalize the returned immunization information to the patient/consumer via the web-based scheduler application or other communication methods.

Client 205 may communicate the one or more immunization opportunities based at least in part on the determining. For example, if there are one or more immunization opportunities available to the patient/consumer, client 205 may provide the patient/consumer (e.g., via the web-based scheduler application or via user device contact 210) the option to either add the registry-recommended one or more immunizations to any existing scheduled provider appointment or to schedule a new appointment for the registry recommended one or more immunizations. In some cases, client 205 may provide access to an online ‘Health and Wellness, scheduling tool so that that the patient/consumer can schedule an appointment for an immunization.

A third non-limiting example of a system for immunization registry integration comprises a digital immunization forms/document-based query. Client 205 or immunization registry/services partner software system 212 may provide access to an immunization services application. For example, patient/consumer may accesses a provider sponsored (e.g., health care provider or immunization registry/services provider) or third-party sponsored website with the intent to learn about immunizations or services provided by the provider. In the body of the webpage accessed by the patient/consumer, client 205 or immunization registry/services partner software system 212 may imbed a URL (e.g., provided by an entity associated with client 205).

Client 205 may receive authorization to query an immunization registry based at least on the provided access. For example, patient/consumer may indicate on the webpage that he or she is interested in accessing, completing, and submitting necessary forms related to immunizations and associated immunization services. Client 205 may receive information associated with the immunization registry based at least in part on the received authorization. For example, the URL may facilitate collection of patient/consumer demographic data elements. For example, client 205 may collect the patient/consumer demographic data elements (i.e., in accordance with relevant privacy regulations) as discussed herein. In some cases, the URL may facilitate collection of a minimum required patient/consumer demographic data elements. The minimum required patient/consumer demographic data elements may constitute one or more of these patient/consumer demographic data elements. Using the data provided by the consumer and collected by client 205 (and/or immunization registry/services partner software system 212), client 205 may reformat the data into an electronic file and transmits that data via an API or sFTP.

Client 205 may determine one or more immunization opportunities based at least in part on the received information associated with immunization registry. In some cases, immunization registry/services partner software system 212 captures and interprets the data provided via the API or sFTP. The immunization registry/services partner software system 212 may access the appropriate local, state or Federal registry (e.g., based at least in part on the demographic data of the provider or patient/consumer). Client 205 and/or immunization registry/services partner software system 212 may match the submitted patient/consumer demographic data with that currently populated/present at the local, state, or Federal registry. For example, if there is a one to one match of patient/consumer demographic data, the immunization registry/services partner software system 212, using associated processes and protocols may return to client 205 via the same sFTP or API a ‘History with Forecast Response,’ which may include one or more reported vaccines administered to the patient/consumer and any CDC recommended (i.e., gap) vaccines that the patient/consumer should consider. If there is not a patient/consumer match or greater than one patient/consumer demographic data set matches the demographic set information submitted by client 205, the immunization registry/services partner software system 212 may return via the same sFTP or API a listing of general vaccines recommended for the patient/consumer according to the CDC based on certain demographic information (e.g., for the gender and age submitted). In some cases, client 205 may reformat the electronic data file information provided by the immunization registry/services partner software system 212. In some cases, client 205 may personalize the returned immunization information to the patient/consumer via the web-based scheduler application or other communication methods.

Client 205 may communicate the one or more immunization opportunities based at least in part on the determining. For example, if there are one or more immunization opportunities available to the patient/consumer, client 205 may provide the patient/consumer (e.g., via the web-based scheduler application or via user device contact 210) the option to either add the registry-recommended one or more immunizations to any existing scheduled provider appointment or to schedule a new appointment for the registry recommended one or more immunizations. In some cases, client 205 may provide access to an online ‘Health and Wellness, scheduling tool so that that the patient/consumer can schedule an appointment for an immunization.

In accordance with the aspects discussed herein, client 205 and/or immunization registry/services partner software system 212 may comprise web-based applications, URLs, APIs, sFTPs, email applications and servers, etc. for providing a system for immunization registry integration that supports vaccine notification and scheduling in automated electronic delivery format. In some implementations, client 205 may facilitate integration and interrelationship with immunization registry/services integration entities (e.g., with remote system contacts 112 and/or immunization registry/services partner software system 212). In some implementations, client 205 may facilitate integration and interrelationship with health and wellness provider entities (e.g., remote system contacts 112).

It should be noted that the first through third non-limiting examples described herein describe possible implementations, and that the operations and the steps may be rearranged or otherwise modified (e.g., performed by different systems and/or entities) and that other implementations are possible. Furthermore, aspects from two or more of the examples may be combined into other implementations.

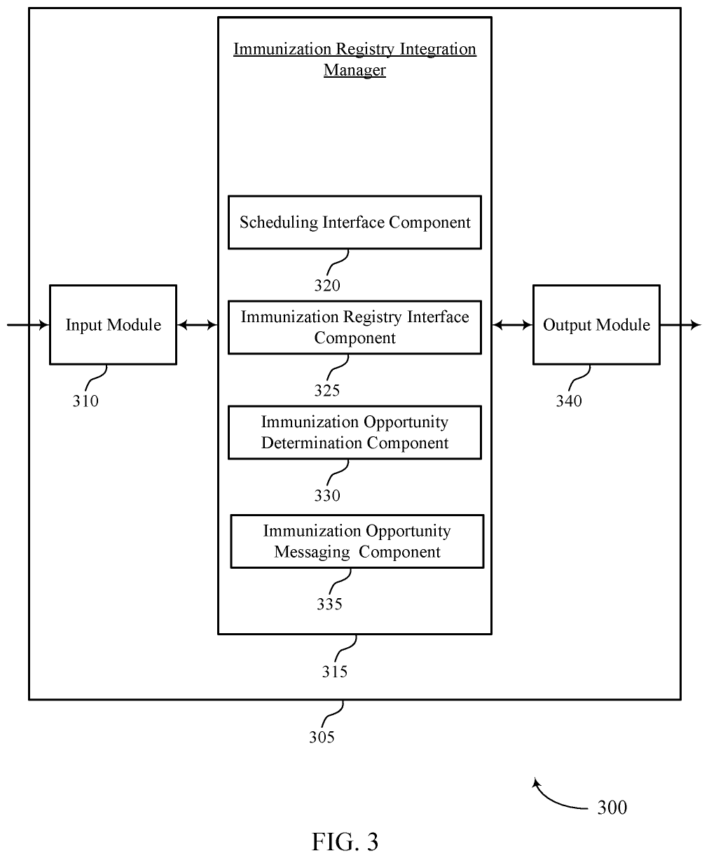

FIG. 3 shows a block diagram of an apparatus associated with a system for immunization registry integration that supports vaccine notification and scheduling in automated electronic delivery format system in accordance with aspects of the present disclosure. The apparatus 305 may include an input module 310, an immunization registry integration manager 315, and an output module 340. The apparatus 305 may also include a processor. Each of these components may be in communication with one another (e.g., via one or more buses). In some cases, the apparatus 305 may be an example of a user terminal, a database server, or a system containing multiple computing devices.

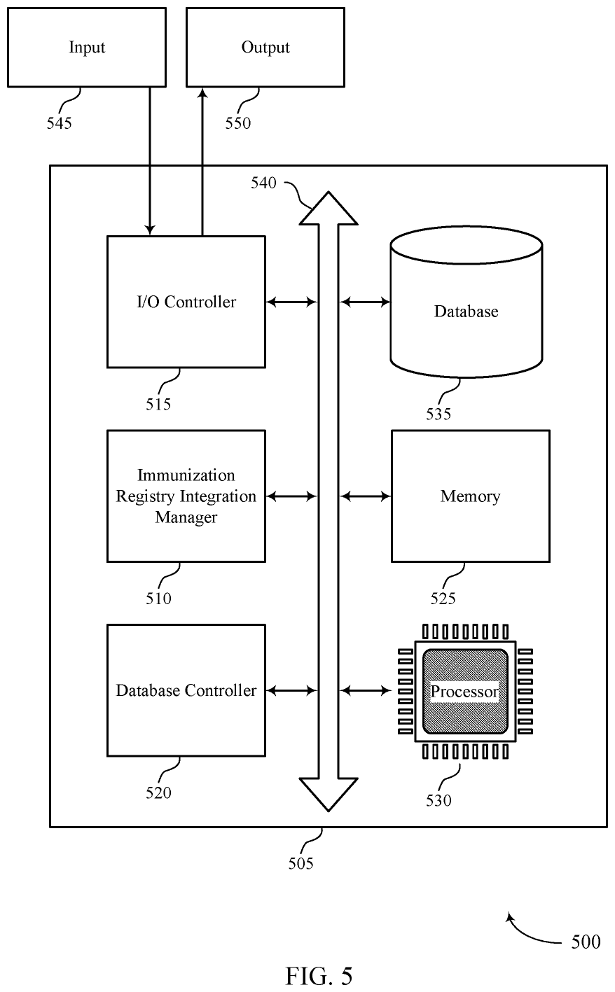

The input module 310 may manage input signals for the apparatus 305. For example, the input module 310 may identify input signals based on an interaction with a modem, a keyboard, a mouse, a touchscreen, or a similar device. These input signals may be associated with user input or processing at other components or devices. In some cases, the input module 310 may utilize an operating system such as iOS®, ANDROID®, MS-DOS®, MS-WINDOWS®, OS/2®, UNIX®, LINUX®, or another known operating system to handle input signals. The input module 310 may send aspects of these input signals to other components of the apparatus 305 for processing. For example, the input module 310 may transmit input signals to the immunization registry integration manager 315 to support a system for immunization registry integration that supports vaccine notification and scheduling in automated electronic delivery format. In some cases, the input module 310 may be a component of an input/output (I/O) controller 515 as described with reference to FIG. 5.

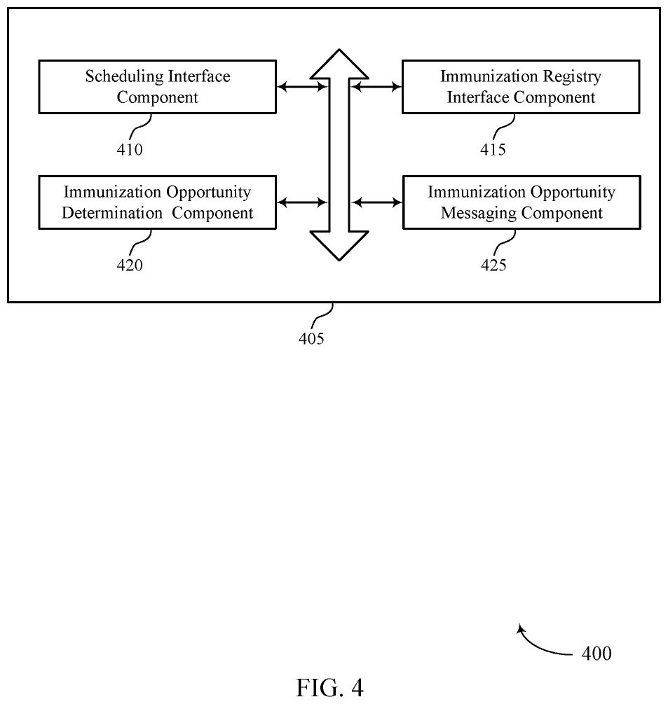

The immunization registry integration manager 315 may include a scheduling interface component 320, an immunization registry interface component 325, an immunization opportunity determination component 330, and an immunization opportunity messaging component 335. The immunization registry integration manager 315 may be an example of aspects of the immunization registry integration manager 405 or 510 described with reference to FIGS. 4 and 5.

The immunization registry integration manager 315 and/or at least some of its various sub-components may be implemented in hardware, software executed by a processor, firmware, or any combination thereof. If implemented in software executed by a processor, the functions of the immunization registry integration manager 315 and/or at least some of its various sub-components may be executed by a general-purpose processor, a digital signal processor (DSP), an application-specific integrated circuit (ASIC), a field-programmable gate array (FPGA) or other programmable logic device, discrete gate or transistor logic, discrete hardware components, or any combination thereof designed to perform the functions described in the present disclosure. The immunization registry integration manager 315 and/or at least some of its various sub-components may be physically located at various positions, including being distributed such that portions of functions are implemented at different physical locations by one or more physical devices. In some examples, the immunization registry integration manager 315 and/or at least some of its various sub-components may be a separate and distinct component in accordance with various aspects of the present disclosure. In other examples, the immunization registry integration manager 315 and/or at least some of its various sub-components may be combined with one or more other hardware components, including but not limited to an I/O component, a transceiver, a network server, another computing device, one or more other components described in the present disclosure, or a combination thereof in accordance with various aspects of the present disclosure.

The scheduling interface component 320 may provide access to a scheduling application to schedule a patient appointment. In some cases, the scheduling interface component 320 may provide access to an immunization services application. In some cases, the scheduling interface component 320 may transmit an indication of a scheduled patient appointment to a device.

The immunization registry interface component 325 may receive authorization to query an immunization registry based at least on the provided access. In some cases, the immunization registry interface component 325 may receive information associated with immunization registry responsive to the transmitted indication. In some cases, the immunization registry interface component 325 may receive information associated with the immunization registry based at least in part on the received authorization.

The immunization opportunity determination component 330 may determine one or more immunization opportunities based at least in part on the received authorization. In some cases, the immunization opportunity determination component 330 may determine one or more immunization opportunities based at least in part on the received information associated with immunization registry.

The immunization opportunity messaging component 335 may communicate the one or more immunization opportunities based at least in part on the determining.

The output module 340 may manage output signals for the apparatus 305. For example, the output module 340 may receive signals from other components of the apparatus 305, such as the immunization registry integration manager 315, and may transmit these signals to other components or devices. In some specific examples, the output module 340 may transmit output signals for display in a user interface, for storage in a database or data store, for further processing at a server or server cluster, or for any other processes at any number of devices or systems. In some cases, the output module 340 may be a component of an I/O controller 515 as described with reference to FIG. 5.

FIG. 4 shows a block diagram of an immunization registry integration manager that supports vaccine notification and scheduling in automated electronic delivery format in accordance with aspects of the present disclosure. The immunization registry integration manager 405 may be an example of aspects of an immunization registry integration manager 315 or an immunization registry integration manager 510 described herein. The immunization registry integration manager 405 may include a scheduling interface component 410, an immunization registry interface component 415, an immunization opportunity determination component 420, and an immunization opportunity messaging component 425. Each of these modules may communicate, directly or indirectly, with one another (e.g., via one or more buses).

The scheduling interface component 410 may provide access to a scheduling application to schedule a patient appointment, may provide access to an immunization services application, may transmit an indication of a scheduled patient appointment to a device, or any combination thereof.

The immunization registry interface component 415 may receive authorization to query an immunization registry based at least on the provided access, may receive information associated with immunization registry responsive to the transmitted indication, may receive information associated with the immunization registry based at least in part on the received authorization, or any combination thereof.

The immunization opportunity determination component 420 may determine one or more immunization opportunities based at least in part on the received authorization, may determine one or more immunization opportunities based at least in part on the received information associated with immunization registry, or any combination thereof.

The immunization opportunity messaging component 425 may communicate the one or more immunization opportunities based at least in part on the determining.

FIG. 5 shows a diagram of a system including a device associated with a system for immunization registry integration that supports vaccine notification and scheduling in automated electronic delivery format in accordance with aspects of the present disclosure. The device 505 may be an example of or include the components of a database server or an apparatus 305 as described herein. The device 505 may include components for bi-directional data communications including components for transmitting and receiving communications, including an immunization registry integration manager 510, an I/O controller 515, a database controller 520, memory 525, a processor 530, and a database 535. These components may be in electronic communication via one or more buses (e.g., bus 540).

The immunization registry integration manager 510 may be an example of an immunization registry integration manager 315 or 405 as described herein. For example, the immunization registry integration manager 510 may perform any of the methods or processes described above with reference to FIGS. 3 and 4. In some cases, the immunization registry integration manager 510 may be implemented in hardware, software executed by a processor, firmware, or any combination thereof.

The I/O controller 515 may manage input signals 545 and output signals 550 for the device 505. The I/O controller 515 may also manage peripherals not integrated into the device 505. In some cases, the I/O controller 515 may represent a physical connection or port to an external peripheral. In some cases, the I/O controller 515 may utilize an operating system such as iOS®, ANDROID®, MS-DOS®, MS-WINDOWS®, OS/2®, UNIX®, LINUX®, or another known operating system. In other cases, the I/O controller 515 may represent or interact with a modem, a keyboard, a mouse, a touchscreen, or a similar device. In some cases, the I/O controller 515 may be implemented as part of a processor. In some cases, a user may interact with the device 505 via the I/O controller 515 or via hardware components controlled by the I/O controller 515.

The database controller 520 may manage data storage and processing in a database 535. In some cases, a user may interact with the database controller 520. In other cases, the database controller 520 may operate automatically without user interaction. The database 535 may be an example of a single database, a distributed database, multiple distributed databases, a data store, a data lake, or an emergency backup database.

Memory 525 may include random-access memory (RAM) and read-only memory (ROM). The memory 525 may store computer-readable, computer-executable software including instructions that, when executed, cause the processor to perform various functions described herein. In some cases, the memory 525 may contain, among other things, a basic input/output system (BIOS) which may control basic hardware or software operation such as the interaction with peripheral components or devices.

The processor 530 may include an intelligent hardware device, (e.g., a general-purpose processor, a DSP, a central processing unit (CPU), a microcontroller, an ASIC, an FPGA, a programmable logic device, a discrete gate or transistor logic component, a discrete hardware component, or any combination thereof). In some cases, the processor 530 may be configured to operate a memory array using a memory controller. In other cases, a memory controller may be integrated into the processor 530. The processor 530 may be configured to execute computer-readable instructions stored in a memory 525 to perform various functions (e.g., functions or tasks supporting a system for immunization registry integration that supports vaccine notification and scheduling in automated electronic delivery format).

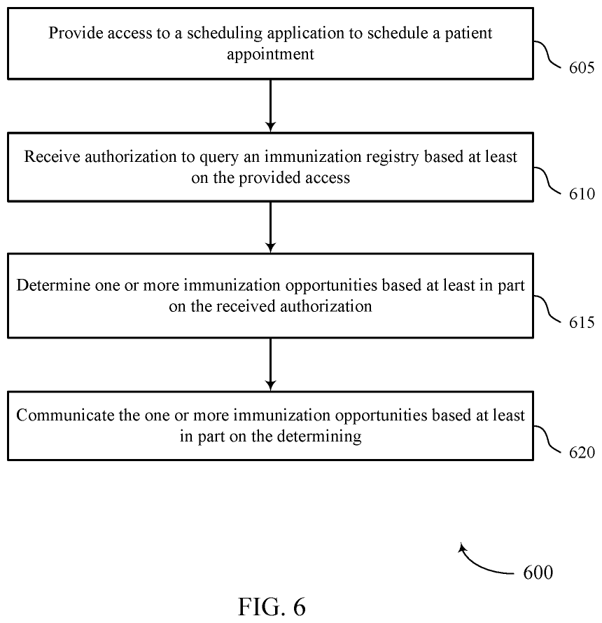

FIG. 6 shows a flowchart illustrating a method 600 associated with a system for immunization registry integration that supports vaccine notification and scheduling in automated electronic delivery format in accordance with aspects of the present disclosure. The operations of method 600 may be implemented by a database server or its components as described herein. For example, the operations of method 600 may be performed by an immunization registry integration manager as described with reference to FIGS. 3 through 5. In some examples, a database server associated with a server or system for immunization registry integration may execute a set of instructions to control the functional elements of the database server to perform the functions described below. Additionally or alternatively, a database server may perform aspects of the functions described below using special-purpose hardware.

At 605, the database server may provide access to a scheduling application to schedule a patient appointment. The operations of 605 may be performed according to the methods described herein. In some examples, aspects of the operations of 605 may be performed by a scheduling interface component as described with reference to FIGS. 3 through 5.

In some non-limiting examples, the database server may receive, over a network, a request to schedule the patient appointment. For example, a user may navigate a web page to a portion of the web page designated for scheduling the patient appointment. The portion of the web page may include graphical elements including pictures, icons, text, and other visual representations as well as HyperText Markup Language (HTML) features (e.g., hyperlinks) and the like. The user may click on the portion of the web page indicating a request to schedule the patient appointment, and responsive to the request a new web page may be initiated by the database server.

At 610, the database server may receive authorization to query an immunization registry based at least on the provided access. The operations of 610 may be performed according to the methods described herein. In some examples, aspects of the operations of 610 may be performed by an immunization registry interface component as described with reference to FIGS. 3 through 5.

In some non-limiting examples, the authorization to query an immunization registry may be received via patient interaction. That is, the authorization to query the immunization registry may be based on the provided access and actions performed by a user or patient while accessing the scheduling application.

At 615, the database server may determine one or more immunization opportunities based at least in part on the received authorization. The operations of 615 may be performed according to the methods described herein. In some examples, aspects of the operations of 615 may be performed by an immunization opportunity determination component as described with reference to FIGS. 3 through 5.

In some non-limiting examples, the database server may obtain one or more data elements based on the request to schedule the patient appointment. For example, one or more data elements may correspond to information such as, but not limited to name, date of birth, gender, address, etc. In some cases, a geographical location identifier may include an identifier for designation a state (e.g., South Carolina, Texas, California, etc.) or a country (e.g., US, Mexico, Canada, UK, etc.). In other cases, the geographical location identifier may include a zip code. In some cases, a plurality of phone numbers may include a string of one or more phone numbers that is used to query the immunization registry. The string of one or more phone numbers may include a first number that the patient entered and a second number determined from a source different from the scheduling application. For example, the source different from the scheduling application may be a secondary source like a patient database managed by a pharmacy or medical practice that is referenced by the scheduling application. At least some of this information may be compared to like information in an immunization registry.

The database server may query the immunization registry based on the one or more data elements. In some cases, the query by the database server may be on behalf of a pharmacy partner, a health care provider partner, or the like. In some cases, the database server determines the one or more immunization opportunities based on the querying. In some cases, the database server may receive from the immunization registry server all of the vaccines that a person has had. In some cases, the database server may utilize CDC guidelines (e.g., access stored information or access a remote server on which CDC guidelines are stored) to determine vaccines that should be administered, if any, that a person based on some of the patient information should have or for which the patient is eligible.

In some non-limiting examples, the database server may determine one or more immunization opportunities that include an immunization opportunity unrelated to the patient appointment. For example, the patient may be scheduling an appointment for a flu shot, and the immunization opportunity may concern a Tetanus or Hepatitis B vaccination. In other examples, the patient may be scheduling an appointment for a physical health examination, and the immunization opportunity may concern a flu shot or a shingles shot. In other examples, the patient may be scheduling an appointment for an eye appointment/vision examination, and the immunization opportunity may concern a flu shot or a shingles shot.

In some non-limiting examples, the database server may determine one or more immunization opportunities that include an immunization opportunity corresponding to the patient appointment. For example, the patient may be scheduling an appointment for a flu shot, and the immunization opportunity may concern a follow-up flu shot (e.g., a second or a two-part flu shot) or a flu shot for next year or next known flu season.

At 620, the database server may communicate the one or more immunization opportunities based at least in part on the determining. The operations of 620 may be performed according to the methods described herein. In some examples, aspects of the operations of 620 may be performed by an immunization opportunity messaging component as described with reference to FIGS. 3 through 5.

In some non-limiting examples, if a vaccination gap is identified in the patient's vaccination records and one or more vaccinations that should be administered, the database server may communicate this information. For example, the database server may provide a user prompt to select the one or more immunization opportunities. In some cases, the database server may provide an opportunity to schedule one or more vaccinations of the identified vaccination gap. For example, the database server may provide access to a new appointment instance of the scheduling application to schedule the one or more immunization opportunities.

In some non-limiting examples, the database server may communicate via a real-time user prompt to add the one or more immunization opportunities to the patient appointment being scheduled. In some cases, the database server may communicate the one or more immunization opportunities via an electronic communication (e.g., a text message or email) subsequent to the patient appointment being scheduled.

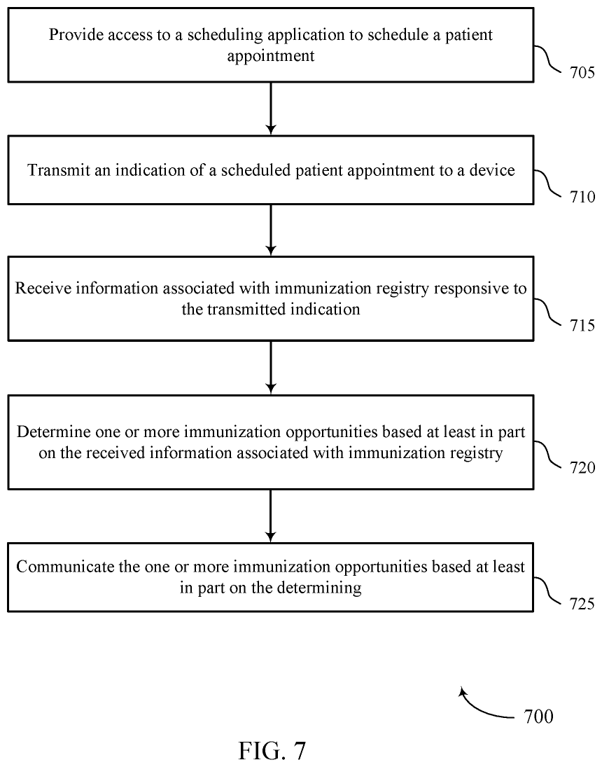

FIG. 7 shows a flowchart illustrating a method 700 associated with a system for immunization registry integration that supports vaccine notification and scheduling in automated electronic delivery format in accordance with aspects of the present disclosure. The operations of method 700 may be implemented by a database server or its components as described herein. For example, the operations of method 700 may be performed by an immunization registry integration manager as described with reference to FIGS. 3 through 5. In some examples, a database server associated with a server or system for immunization registry integration may execute a set of instructions to control the functional elements of the database server to perform the functions described below. Additionally or alternatively, a database server may perform aspects of the functions described below using special-purpose hardware.

At 705, the database server may provide access to a scheduling application to schedule a patient appointment. The operations of 705 may be performed according to the methods described herein. In some examples, aspects of the operations of 705 may be performed by a scheduling interface component as described with reference to FIGS. 3 through 5.

At 710, the database server may transmit an indication of a scheduled patient appointment to a device. The operations of 710 may be performed according to the methods described herein. In some examples, aspects of the operations of 710 may be performed by a scheduling interface component as described with reference to FIGS. 3 through 5.

At 715, the database server may receive information associated with immunization registry responsive to the transmitted indication. The operations of 715 may be performed according to the methods described herein. In some examples, aspects of the operations of 715 may be performed by an immunization registry interface component as described with reference to FIGS. 3 through 5.

At 720, the database server may determine one or more immunization opportunities based at least in part on the received information associated with immunization registry. The operations of 720 may be performed according to the methods described herein. In some examples, aspects of the operations of 720 may be performed by an immunization opportunity determination component as described with reference to FIGS. 3 through 5.

At 725, the database server may communicate the one or more immunization opportunities based at least in part on the determining. The operations of 725 may be performed according to the methods described herein. In some examples, aspects of the operations of 725 may be performed by an immunization opportunity messaging component as described with reference to FIGS. 3 through 5.

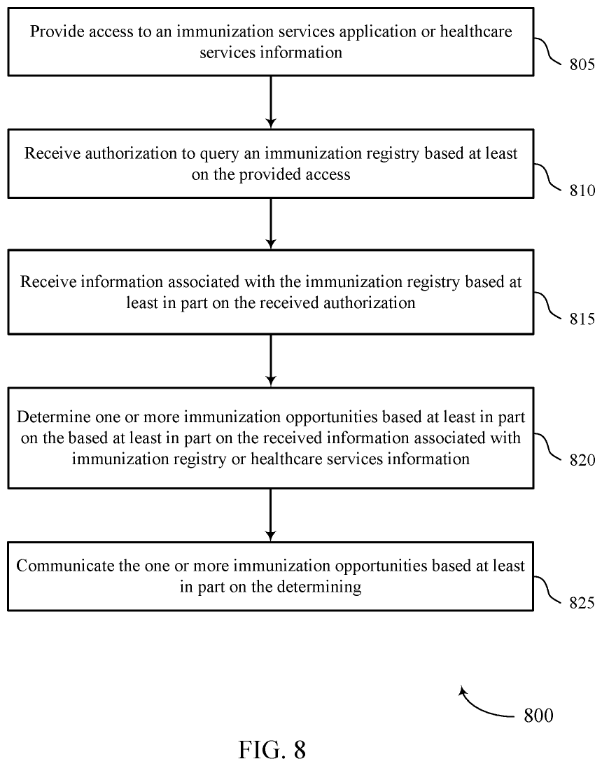

FIG. 8 shows a flowchart illustrating a method 800 associated with a system for immunization registry integration that supports vaccine notification and scheduling in automated electronic delivery format in accordance with aspects of the present disclosure. The operations of method 800 may be implemented by a database server or its components as described herein. For example, the operations of method 800 may be performed by an immunization registry integration manager as described with reference to FIGS. 3 through 5. In some examples, a database server associated with a server or system for immunization registry integration may execute a set of instructions to control the functional elements of the database server to perform the functions described below. Additionally or alternatively, a database server may perform aspects of the functions described below using special-purpose hardware.

At 805, the database server may provide access to an immunization services application or healthcare services information. The operations of 805 may be performed according to the methods described herein. In some examples, aspects of the operations of 805 may be performed by a scheduling interface component as described with reference to FIGS. 3 through 5.

In some non-limiting examples, the database server may identify information from a medical-related digital document based at least in part on provided access. For example, the medical-related digital document may be an immunization or other health-related digital consent and information form. For example, the medical-related digital document may include a questionnaire. That is, the question regarding whether the patient has a fever, any pains, etc., along with other information such as but not limited to name, date of birth, gender, address, etc. In some cases, the database server may receive, over a network, a request to provide the medical-related digital document. In other cases, the database server may receive, over a network, the medical-related digital document, for example from a pharmacy partner, a health care provider partner, or the like.

At 810, the database server may receive authorization to query an immunization registry based at least on the provided access. The operations of 810 may be performed according to the methods described herein. In some examples, aspects of the operations of 810 may be performed by an immunization registry interface component as described with reference to FIGS. 3 through 5.

At 815, the database server may receive information associated with the immunization registry based at least in part on the received authorization. The operations of 815 may be performed according to the methods described herein. In some examples, aspects of the operations of 815 may be performed by an immunization registry interface component as described with reference to FIGS. 3 through 5.

At 820, the database server may determine one or more immunization opportunities based at least in part on the received information associated with immunization registry or healthcare services information. The operations of 820 may be performed according to the methods described herein. In some examples, aspects of the operations of 820 may be performed by an immunization opportunity determination component as described with reference to FIGS. 3 through 5.

In some non-limiting examples, the database server may obtain one or more data elements based on information from the medical-related digital document. For example, one or more data elements may correspond to information such as but not limited to name, date of birth, gender, address, etc. At least some of this information may be compared to like information in an immunization registry.

At 825, the database server may communicate the one or more immunization opportunities based at least in part on the determining. The operations of 825 may be performed according to the methods described herein. In some examples, aspects of the operations of 825 may be performed by an immunization opportunity messaging component as described with reference to FIGS. 3 through 5.

It should be noted that the methods described above describe possible implementations, and that the operations and the steps may be rearranged or otherwise modified and that other implementations are possible. Furthermore, aspects from two or more of the methods may be combined.

The following examples are given by way of illustration. Aspects of the following examples may be combined with aspects or embodiments shown or discussed in relation to the figures or elsewhere herein.

Aspect 1 is a method for immunization registry integration, comprising: providing access to a scheduling application to schedule a patient appointment; receiving authorization to query an immunization registry based at least on the provided access; determining one or more immunization opportunities based at least in part on the received authorization; and communicating the one or more immunization opportunities based at least in part on the determining.

In Aspect 2, the method of Aspect 1 may further comprise receiving, over a network, a request to schedule the patient appointment. In Aspect 2, the providing access to the scheduling application may be responsive to the request.

In Aspect 3, the method of Aspect 2 may further comprise: obtaining one or more data elements based at least in part on the request to schedule the patient appointment; and querying the immunization registry based at least in part on the one or more data elements. In Aspect 3, the determining one or more immunization opportunities may be based at least in part on the querying.

In Aspect 4, the one or more data elements in Aspect 3 may comprise an identifier corresponding to at least one of name, date of birth, or gender.

In Aspect 5, the one or more data elements in any of Aspect 3 or 4 may comprise a geographical location identifier.

In Aspect 6, the one or more data elements in any of Aspects 3-5 may comprise a plurality of phone numbers.

In Aspect 7, the plurality of phone numbers in Aspect 6 may comprise a first phone number entered for scheduling the patient appointment and a second phone number determined from a source different from the scheduling application.

In Aspect 8, the one or more immunization opportunities in any of Aspects 1-7 may comprise an immunization opportunity unrelated to the patient appointment.

In Aspect 9, the one or more immunization opportunities in any of Aspects 1-7 may comprise an immunization opportunity corresponding to the patient appointment.

In Aspect 10, the method of any of Aspects 1-9 may further comprise providing a user prompt to select the one or more immunization opportunities based at least in part on the communicating the one or more immunization opportunities.

In Aspect 11, the method of any of Aspects 1-10 may further comprise providing access to a new appointment instance of the scheduling application to schedule the one or more immunization opportunities based at least in part on the communicating the one or more immunization opportunities.

In Aspect 12, the communicating the one or more immunization opportunities in any of Aspects 1-11 may comprise a real-time user prompt to add the one or more immunization opportunities to the patient appointment being scheduled.

In Aspect 13, the communicating the one or more immunization opportunities in any of Aspects 1-12 may comprise an electronic communication subsequent to the patient appointment being scheduled.

Aspect 14 is a method for immunization registry integration, comprising: providing access to healthcare services information; identifying information from a medical-related digital document based at least in part on provided access; receiving authorization to query an immunization registry based at least on the medical-related digital document; determining one or more immunization opportunities based at least in part on the received authorization; and communicating the one or more immunization opportunities based at least in part on the determining.

In Aspect 15, the method of Aspect 14 may comprise a receiving, over a network, a request to provide the medical-related digital document.

In Aspect 16, the method of Aspects 14 or 15 may further comprise receiving, over a network, the medical-related digital document.

In Aspect 17, the method of any of Aspects 14-16 may further comprise: obtaining one or more data elements based at least in part on the information from the medical-related digital document; and querying the immunization registry based at least in part on the one or more data elements. In Aspect 17, the determining one or more immunization opportunities may be based at least in part on the querying.

In Aspect 18, the method of any of Aspects 14-17 may further comprise providing a user prompt to select the one or more immunization opportunities based at least in part on the communicating the one or more immunization opportunities.

In Aspect 19, the method of any of Aspects 14-18 may further comprise providing access to a scheduling application to schedule the one or more immunization opportunities based at least in part on the communicating the one or more immunization opportunities.

In Aspect 20, the medical-related digital document in any of Aspects 14-19 may comprise a response to at least one health-related question.

Aspect 21 is a system, server, or apparatus including one or more processors and memory operatively coupled with the one or more processors storing instructions executable by the one or more processors to cause the system, server, or apparatus to implement a method as in any of Aspects 1-13.

Aspect 22 is a system, server, or apparatus including means for implementing a method or realizing a system, server, or apparatus as in any of Aspects 1-13.

Aspect 23 is a non-transitory computer-readable medium storing instructions executable by one or more processors to cause the one or more processors to implement a method as in any of aspects 1-13.

Aspect 24 is a system, server, or apparatus including one or more processors and memory operatively coupled with the one or more processors storing instructions executable by the one or more processors to cause the system, server, or apparatus to implement a method as in any of Aspects 14-20.

Aspect 25 is a system, server, or apparatus including means for implementing a method or realizing a system, server, or apparatus as in any of Aspects 14-20.

Aspect 26 is a non-transitory computer-readable medium storing instructions executable by one or more processors to cause the one or more processors to implement a method as in any of aspects 14-20.

Examples of these aspects may be combined with aspects or embodiments disclosed in other implementations.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable read only memory (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

1. A method for immunization registry integration by a first computing device, comprising:
providing, to a user device, access to a scheduling application to schedule a patient appointment of a first type;
receiving, from the user device, authorization to query an immunization registry based at least on the provided access;
determining one or more immunization opportunities of a type different from the first type based at least in part on the received authorization and a file transfer protocol or programming interface associated with a second computing device different from the first computing device; and
communicating, to the user device, the one or more immunization opportunities based at least in part on the determining.

2. The method of claim 1, further comprising:
receiving, over a network, a request to schedule the patient appointment, and
wherein the providing access to the scheduling application is responsive to the request.

3. The method of claim 2, further comprising:
obtaining one or more data elements based at least in part on the request to schedule the patient appointment; and
querying the immunization registry based at least in part on the one or more data elements, and
wherein the determining one or more immunization opportunities is based at least in part on the querying.

4. The method of claim 3, wherein the one or more data elements comprise an identifier corresponding to at least one of name, date of birth, or gender.

5. The method of claim 3, wherein the one or more data elements comprise a geographical location identifier.

6. The method of claim 3, wherein the one or more data elements comprise a plurality of phone numbers.

7. The method of claim 6, wherein the plurality of phone numbers comprises a first phone number entered for scheduling the patient appointment and a second phone number determined from a source different from the scheduling application.

8. The method of claim 1, wherein the one or more immunization opportunities comprises an immunization opportunity unrelated to the patient appointment.

9. The method of claim 1, wherein the one or more immunization opportunities comprises an immunization opportunity corresponding to the patient appointment.

10. The method of claim 1, further comprising:
providing a user prompt to select the one or more immunization opportunities based at least in part on the communicating the one or more immunization opportunities.

11. The method of claim 1, further comprising:
providing access to a new appointment instance of the scheduling application to schedule the one or more immunization opportunities based at least in part on the communicating the one or more immunization opportunities.

12. The method of claim 1, wherein the communicating the one or more immunization opportunities comprises a real-time user prompt to add the one or more immunization opportunities to the patient appointment being scheduled.

13. The method of claim 1, wherein the communicating the one or more immunization opportunities comprises an electronic communication subsequent to the patient appointment being scheduled.

14. A method for immunization registry integration by a first computing device, comprising:
providing, to a user device, access to healthcare services information;
identifying information of a first type from a medical-related digital document based at least in part on provided access;
receiving, from the user device, authorization to query an immunization registry based at least on the medical-related digital document;
determining one or more immunization opportunities of a type different from the first type based at least in part on the received authorization and a file transfer protocol or programming interface associated with a second computing device different from the first computing device; and
communicating, to the user device, the one or more immunization opportunities based at least in part on the determining.

15. The method of claim 14, further comprising:
receiving, over a network, a request to provide the medical-related digital document.

16. The method of claim 14, further comprising:
receiving, over a network, the medical-related digital document.

17. The method of claim 14, further comprising:
obtaining one or more data elements based at least in part on the information from the medical-related digital document; and
querying the immunization registry based at least in part on the one or more data elements, and
wherein the determining one or more immunization opportunities is based at least in part on the querying.

18. The method of claim 14, further comprising:
providing a user prompt to select the one or more immunization opportunities based at least in part on the communicating the one or more immunization opportunities.

19. The method of claim 14, further comprising:
providing access to a scheduling application to schedule the one or more immunization opportunities based at least in part on the communicating the one or more immunization opportunities.

20. The method of claim 14, wherein the medical-related digital document comprises a response to at least one health-related question.
